(19) United States
(12) Patent Application Publication
(10) Pub. No.: US 2025/0302461 A1
(43) Pub. Date: Oct. 2, 2025

(54) SYSTEM AND METHOD FOR DEPLOYING A HEMOSTATIC IMPLANT IN A TISSUE TRACT

(71) Applicant: Haemonetics Corporation, Boston, MA (US)

(72) Inventor: Jimmy Jen, Saratoga, CA (US)

(21) Appl. No.: 19/089,255

(22) Filed: Mar. 25, 2025

Related U.S. Application Data
(63) Continuation of application No. PCT/US25/20741, filed on Mar. 20, 2025.
(60) Provisional application No. 63/570,339, filed on Mar. 27, 2024.

(51) Int. Cl. A61B 17/00

(52) U.S. Cl. CPC A61B 17/0057; A61B 2017/00623; A61B 2017/00646; A61B 2017/00862; A61B 2017/00893; A61B 2017/00898; A61B 2017/00942

(57) ABSTRACT

An apparatus is provided for sealing a blood vessel wall penetration disposed at an end of a tissue tract. The apparatus, in one embodiment, includes a shaft having a proximal end and a distal end; an occlusion element near the distal end of the shaft; a hemostatic implant disposed over or adjacent to an exterior surface of the shaft proximal to the occlusion element, said hemostatic implant being hydratable to expand to occlude the tissue tract when exposed to body fluids within the tissue tract; a sleeve peelably disposed over an outer surface of the hemostatic implant and proximal to the occlusion element; and wherein peeling of the sleeve exposes the hemostatic implant such that the hemostatic implant expands and occludes the tissue tract.

### RELATED APPLICATIONS

This patent application is a continuation of International Patent Application No. PCT/US2025/020741, filed Mar. 20, 2025, and claims priority from provisional U.S. patent application No. 63/570,339, filed Mar. 27, 2024, naming Jimmy Jen as inventor. The disclosure of each of these applications is incorporated herein, in its entirety, by reference.

### FIELD

Illustrative embodiments of the invention generally relate to medical devices and methods and, more particularly, various embodiments of the invention relate to apparatus and protocols for closing arteriotomies and other vascular wall penetrations.

### BACKGROUND

Angiography, angioplasty, atherectomy, and a number of other vascular and cardiovascular procedures are performed intravascularly and require percutaneous access into the patient's vasculature. The most common technique for achieving percutaneous access is called the Seldinger technique, where access to a femoral vessel is first established using a needle to form a “tract,” i.e., a passage through the tissue overlying the blood vessel. The needle tract is then dilated, and an access sheath is placed into the dilated tract and through a penetration in the vascular wall, such as an arteriotomy or venotomy to allow the introduction of guidewires, interventional catheters, catheter exchange, and the like to perform the desired procedure.

Once the desired procedure is completed, the access sheath must be removed and the arteriotomy or other vascular wall penetration closed. For many years, such closure was achieved by applying manual pressure onto the patient's skin over the site of the vascular wall penetration. Patients, however, have often been heparinized to limit the risk of thrombosis during the procedure, and clotting of the vascular wall penetration can often take an extended period, particularly when the penetration is relatively large for performing procedures needing larger diameter catheters. For these reasons, improved methods for closing and sealing vascular wall penetrations have been sought.

It would be desirable to provide improved methods and systems for deploying hydratable hemostatic implants within a tissue tract in order to achieve closure of vascular wall penetrations. It would be particularly desirable if such methods and devices were compatible with deployment of temporary occlusion elements within the blood vessel lumen, and in particular if such methods and devices reduced the risk of premature implant hydration and swelling which can occur prior to withdrawing the protective sleeve. At least some of these objectives will be met by the disclosures described herein below.

### SUMMARY OF VARIOUS EMBODIMENTS

In accordance with one embodiment of the invention, an apparatus is provided for sealing a blood vessel wall penetration disposed at the distal end of a tissue tract. The apparatus, in one embodiment, includes a shaft having a proximal end and a distal end; a hemostatic implant disposed over or adjacent to an exterior surface of the shaft proximal to the distal end, said hemostatic implant being hydratable to expand to occlude the tissue tract when exposed to body fluids within the tissue tract; and a sleeve peelably disposed over an outer surface of the hemostatic implant and proximal to the distal end. The peeling of the sleeve exposes the hemostatic implant and allows the hemostatic implant to expand to occlude the tissue tract.

A related embodiment includes a shaft having a proximal end and a distal end; an occlusion element near the distal end of the shaft; a hemostatic implant disposed over or adjacent to an exterior surface of the shaft proximal to the occlusion element, said hemostatic implant being hydratable to expand to occlude the tissue tract when exposed to body fluids within the tissue tract; and a sleeve peelably disposed over an outer surface of the hemostatic implant and proximal to the occlusion element; wherein peeling of the sleeve exposes the hemostatic implant such that the hemostatic implant expands and occludes the tissue tract. The sleeve may include one or more longitudinal slits.

In one embodiment, the apparatus further includes a retractable member, wherein the retractable member is attached to a distal end of the sleeve, such that when the retractable member is retracted in a proximal direction, the sleeve is peeled away from the outer surface of the hemostatic implant. In one embodiment, the retractable member is cylindrical and surrounds the hemostatic implant and the sleeve, such that as the retractable member is retracted in a proximal direction the hemostatic implant and the sleeve are exposed. In one embodiment, the retractable member may include a ring-like structure at its distal end.

In one embodiment, the occlusion element is shiftable between a radially contracted configuration for passage through the tissue tract and a radially expanded configuration for deployment within the blood vessel to occlude the penetration. In one embodiment, the apparatus further comprises a back stop on the shaft, wherein the back stop engages the hemostatic implant to immobilize the implant while the sleeve is being peeled from the implant and the contracted occlusion element is being withdrawn past the implant.

In one embodiment, the shaft includes an outer tube and an inner rod, and wherein the occlusion element has a distal end connected to a distal end of the rod and a proximal end connected to a distal end of the tube so that proximal retraction of the rod relative to the tube effects radial expansion of the occlusion element and distal advancement of the rod relative to the tube effects radial contraction of the occlusion element, wherein the occlusion element comprises a braided mesh covered by an elastic membrane.

In one embodiment, the hemostatic implant comprises a body which circumscribes the shaft. The hemostatic implant may comprise a body which is configured to open and expand away laterally from the shaft. The hydratable hemostatic implant preferably includes a biodegradable polymer selected from the group consisting of polyethylene glycols, collagens, and gelatins.

In another embodiment, a method is provided for sealing a blood vessel penetration disposed at the end of a tissue tract. The method includes the steps of providing an apparatus including a shaft, a hemostatic implant disposed on an exterior surface of the shaft, and a sleeve covering outer surfaces of the hemostatic implant; introducing the shaft through the tissue tract to position the hemostatic implant within the tissue tract, wherein the hemostatic implant is covered by the sleeve while the shaft is being introduced; peeling the sleeve to expose the hemostatic implant to the tissue tract, wherein the hemostatic implant expands upon direct contact with the tissue tract; and withdrawing the shaft past the expanded hemostatic implant which remains in the tissue tract.

In one embodiment of the method, the apparatus further includes an occlusion element, and the method further comprises deploying the occlusion element to inhibit blood flow from the blood vessel into the tissue tract. In a preferred embodiment, deploying the occlusion element includes shifting the occlusion element between a radially contracted configuration for passage through the tissue tract and a radially expanded configuration for deployment within the blood vessel to occlude the penetration. The hemostatic implant may be prevented from being displaced proximally by a back stop on the shaft while the sleeve is peeled and while the shaft is withdrawn.

In an embodiment of the method, an introducer or vascular access sheath is used with the sealing apparatus to seal a blood vessel penetration disposed at the end of a tissue tract. The access sheath passes through the tissue tract from a skin surface to the blood vessel. The sealing apparatus includes a shaft, a hemostatic implant disposed on an exterior surface of the shaft, a sleeve covering an outer surface of the hemostatic implant, and an expandable occlusion element. The sealing apparatus is placed within the access sheath and positioned within the tissue tract so that the sealing apparatus protrudes into the blood vessel. The hemostatic implant is covered by the sleeve while the sealing apparatus is within the access sheath and being introduced into the tissue tract. While the sealing apparatus is positioned within the sealing apparatus through the tissue tract, the occlusion element is expanded within the blood vessel and then seated against a wall of the blood vessel at the end of the tissue tract to inhibit blood flow from the blood vessel into the tissue tract. In addition, the access sheath is removed from the tissue tract, leaving the sealing apparatus in the tissue tract. After removing the access sheath, the sleeve is peeled away from the hemostatic implant to expose the hemostatic implant to the tissue tract. Upon exposure to the fluids in the tissue tracts, the hemostatic implant expands upon direct contact with the tissue tract. After the hemostatic implant is exposed to the tissue tract, the occlusion element is contracted, and then the shaft and the occlusion element are withdrawn past the expanded hemostatic implant, which remains in the tissue tract.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 illustrates an example of a prior sealing apparatus, shown in section.

FIG. 1A shows a detail view of the apparatus of FIG. 1.

FIG. 2 is a sectional view of the sealing apparatus of FIG. 1, shown with an expanded occlusion element.

FIGS. 3-7 illustrate the further steps of deployment of the hemostatic implant from the apparatus of FIGS. 1 and 2.

FIGS. 8A-8K illustrate placement and deployment of the hemostatic implant using an apparatus according to one embodiment of the present invention.

FIGS. 9 and 10 are cut-away detail views of an apparatus according to one embodiment of the invention illustrating the beginning stages of the sleeve being peeled away so as to expose the hemostatic implant to the tissue.

FIG. 11 is a perspective detail view of an apparatus corresponding to the arrangement shown in FIG. 9.

FIG. 12 is a cut-away perspective view of an apparatus corresponding to the arrangement shown in FIG. 10.

### DESCRIPTION OF ILLUSTRATIVE EMBODIMENTS

The peelable sleeve of the present invention may be used in systems, such as that shown in U.S. Pat. No. 11,690,608 issued Jul. 4, 2023 to Zia Yassinzadeh (the Yassinzadeh patent), said patent being incorporated herein by reference in its entirety. The Yassinzadeh patent shows a catheter-based delivery system that delivers a hemostatic implant, e.g., a collagen implant, in a tissue tract and adjacent a vascular wall. As discussed in the Background section above, the tissue tract may have been formed for a cardiovascular procedure using a guidewire and/or guidewire that passes into the patient's arterial vasculature, including for example, the femoral artery in the groin.

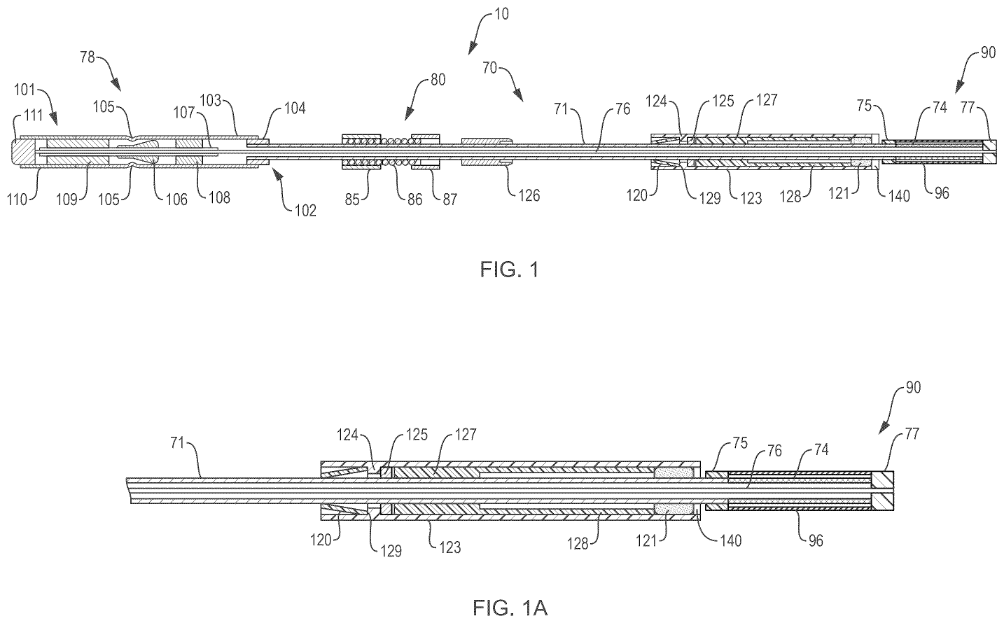

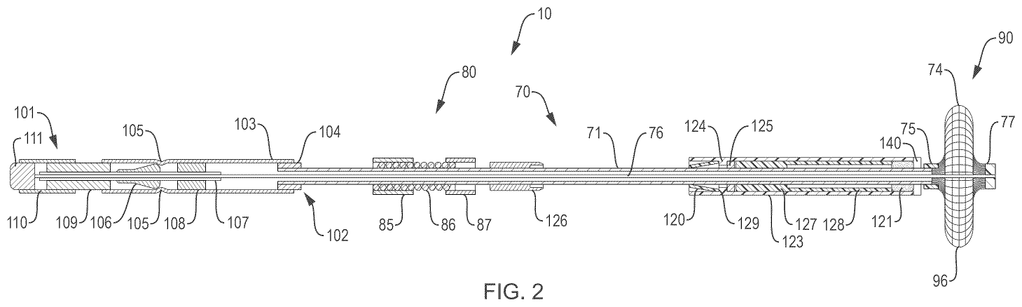

Referring to FIGS. 1 and 1A, a prior exemplary sealing apparatus 10 constructed comprises a shaft assembly 70 including an outer tube 71 and an inner rod 76. An expansible occlusion element 90 is mounted at a distal end (to the right in FIGS. 1 and 1A) of the shaft assembly 70 and includes a radially expansible mesh 74 covered by an elastomeric membrane 96. A handle assembly 78 is attached to a proximal end of the shaft assembly 70 and is operatively attached to both the outer tube 71 and inner rod 76 so that the inner rod can be axially advanced and retracted relative to the outer tube. The inner rod 76 and outer tube 71 are coupled together at the distal tip of the sealing apparatus 10 by a plug 77 and a proximal anchor 75, respectively. The occlusion element 90 is held between the plug 77 and the proximal anchor 75 so that axial retraction of the rod in the proximal direction (to the left as shown in FIGS. 1 and 1A) foreshortens the occlusion element 90, causing the occlusion element to expand radially, as shown for example in FIG. 2. Axial advancement and retraction of the rod 76 relative to the outer tube 71 is effected using the handle assembly 78. The handle assembly 78 includes a cylindrical body 103 attached to the proximal end of the outer tube 71 by a bushing 104 so that the body 103 will remain fixed relative to the outer tube as the inner rod 76 is retracted and advanced. The inner rod is retracted and advanced by a slide assembly 101 which includes a short tube 110 fixedly attached to an endcap 111 and a slide cylinder 109. The inner rod 76 is secured by tube element 107 which carries locking element 106 and bearing elements 108 and 109. Bearing element 109 is attached to proximal grip 101, and the assembly of the grip 101 and tube element 107 can slide freely within the interior of the cylindrical body 103 so that the rod 76 may be proximally retracted relative to the body 103 and outer tube 71, as shown in FIG. 2. Once the expansible occlusion element 90 has been radially expanded, the rod 76 will remain retracted and is held in place by locking element 106 which is pulled over a detent 105, again as shown in FIG. 2. An alignment bushing 108 is provided in the interior of the cylindrical body 103 to maintain alignment of the slide assembly 101 relative to the cylindrical body.

The sealing apparatus may optionally include a tensioning mechanism 80 which includes a coil spring 86, a gripping element 85, and a coupling element 87. The tensioning mechanism 80 may be selectively positioned along the length of shaft assembly 70, and will provide a tension determined by the constant of coil spring 86 to hold the expanded occlusion element 74 against the vascular penetration.

As best seen in FIG. 1A, a hydratable hemostatic implant 121, which will typically be a biodegradable polymer, is carried coaxially or in parallel over the outer tube 71 near the distal end thereof proximal to the expansible occlusion element 90. While the hydratable hemostatic implant 121 is shown to be positioned coaxially over outer tube 71 in FIG. 1A, it will often be desirable to modify or reposition the implant in order to facilitate release from the sealing apparatus after the implant has been deployed. More simply, the hemostatic implant could be axially split to allow it to partially open after it is rehydrated and facilitate passage of the collapsed occlusion element 74 as the sealing apparatus is being withdrawn.

Alternatively, the hemostatic implant may be reconfigured and carried laterally (i.e., to one side of) with respect to the shaft of the sealing apparatus. The hydratable hemostatic implant 121 could alternatively be carried on the inner surface of a protective sleeve 123, which in the prior system is slidably carried over the outer tube 71 and in the present invention is peelably carried over the outer tube. The prior protective sleeve 123 slides over a backstop 127 which is slidably mounted over the outer tube 71 and which is prevented from moving proximally by stop member 125 which is fixed to the outer surface of the outer tube. Backstop 127 has a distal end 128 which engages a proximal end of the hemostatic implant 121. Thus, by proximally retracting the protective sleeve 123, the hydratable hemostatic implant 121 can be exposed to the tissue tract and released from the sealing apparatus. Prior to retraction of the protective sleeve 123, a biodegradable plug 140 protects the hydratable hemostatic implant 121 from exposure to blood or other body fluids when present in the tissue tract. The plug 140 may be composed of any of the materials discussed above, typically being formed from hyaluronic acid, which is highly water soluble.

So long as the hyaluronic acid plug 140 remains beneath the protective sleeve, it will retain sufficient mechanical integrity to block or inhibit passage of significant amounts of fluids to the hydratable hemostatic implant 121. Once the protective sleeve 123 is retracted, however, the hyaluronic acid will quickly absorb water and dissolve in the body fluids, becoming resorbed by the tissue over a relatively short time frame. In contrast, the swollen collagen implant will not dissolve and will be resorbed only slowly over time in order to provide the desired hemostatic effect.

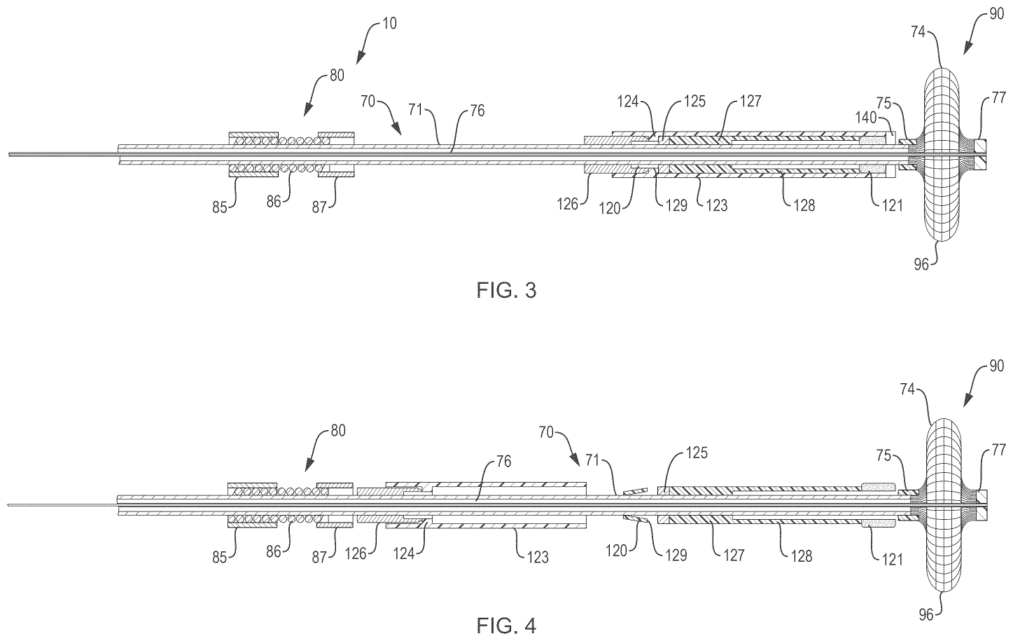

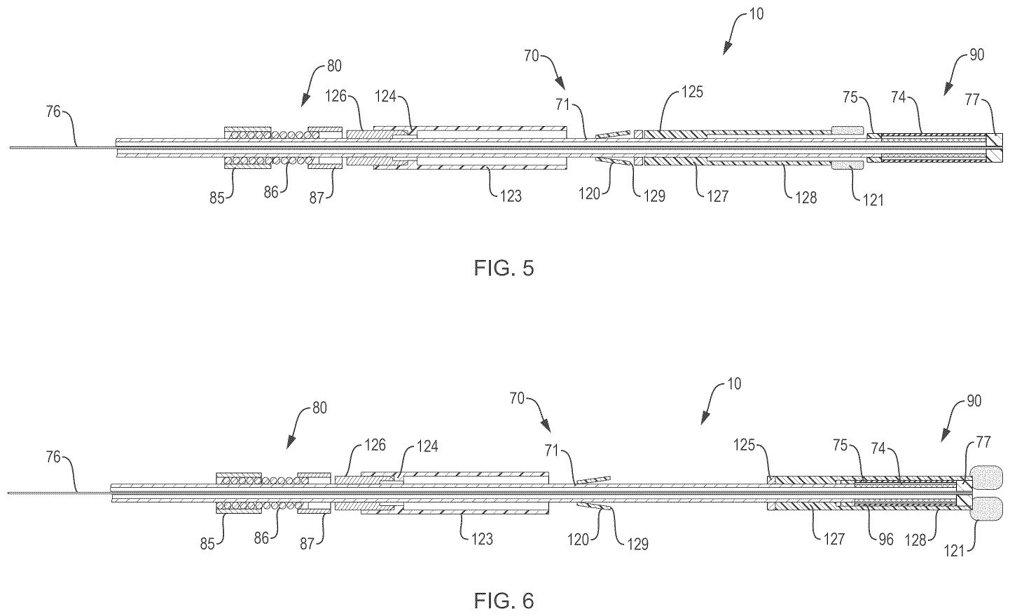

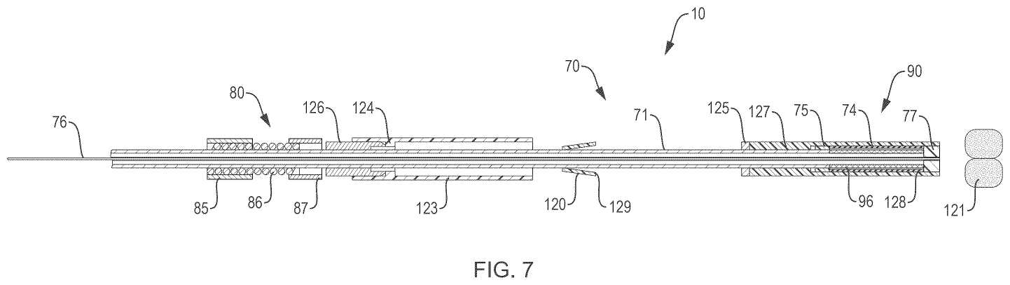

The protective sleeve 123, as shown in FIG. 3, may be proximally withdrawn past the hemostatic implant 121 and the backstop 127, as shown in FIG. 4. Thus, the hemostatic implant 121 will be released from constraint and exposed to the environment in the tissue tract. The environment in the tissue tract will include blood and other body fluids which can hydrate the hemostatic implant 121, causing swelling as shown in FIG. 4. The swelling will continue, as shown in FIG. 5, and the radially expanded occlusion element 90 can be collapsed using the handle assembly, as shown in FIG. 5. The collapsed occlusion element 90 can then be proximally withdrawn into and through the backstop assembly 127, as shown in FIG. 6 (where an annular space may be provided to accommodate the occlusion element). When the occlusion element has been fully withdrawn within the backstop 127, the hemostatic implant is completely released, as shown in FIG. 6, and the remaining portions of the sealing apparatus can be pulled away from the hemostatic implant, as shown in FIG. 7.

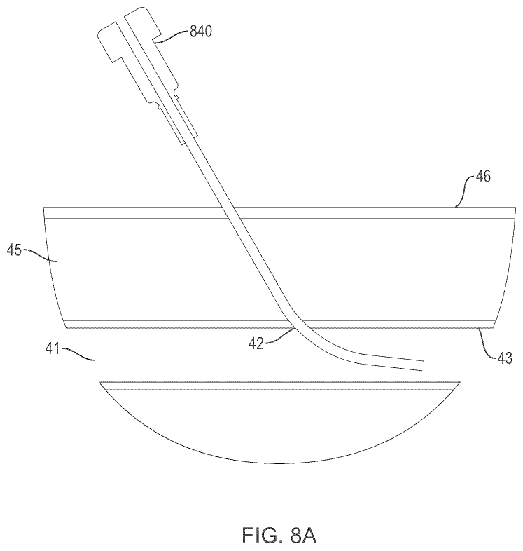

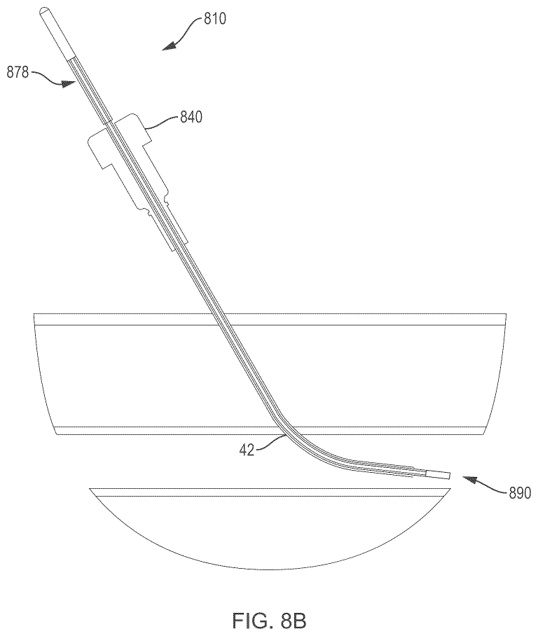

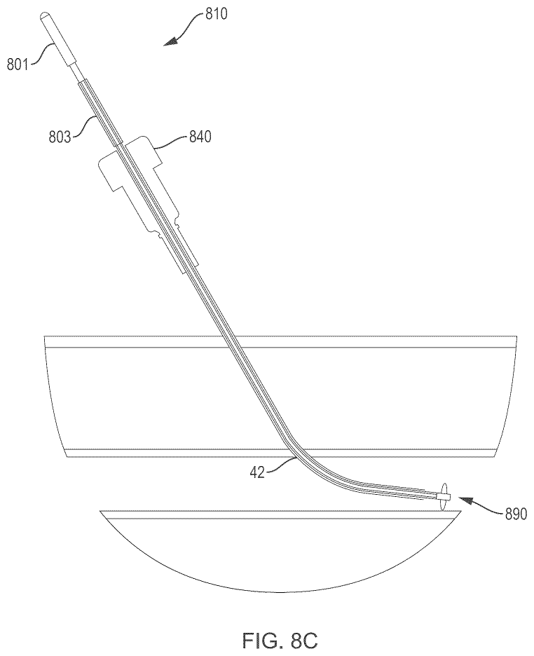

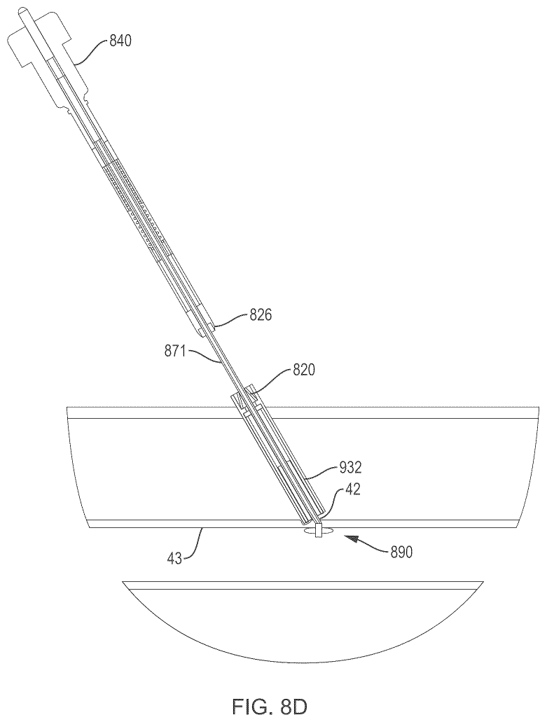

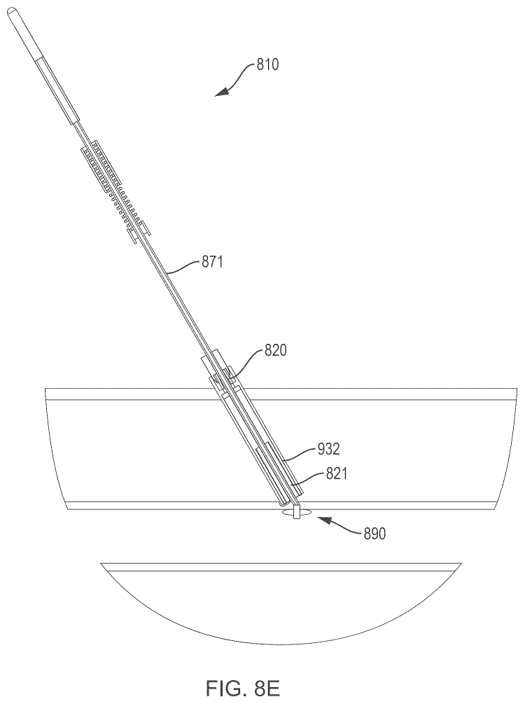

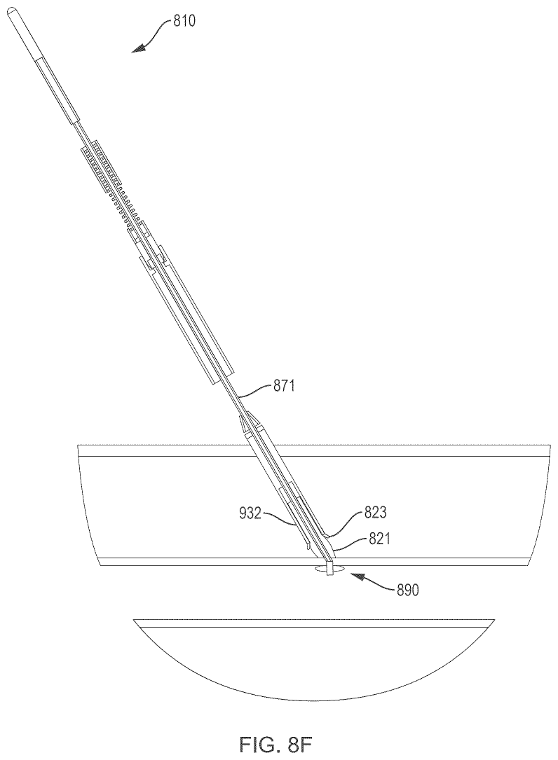

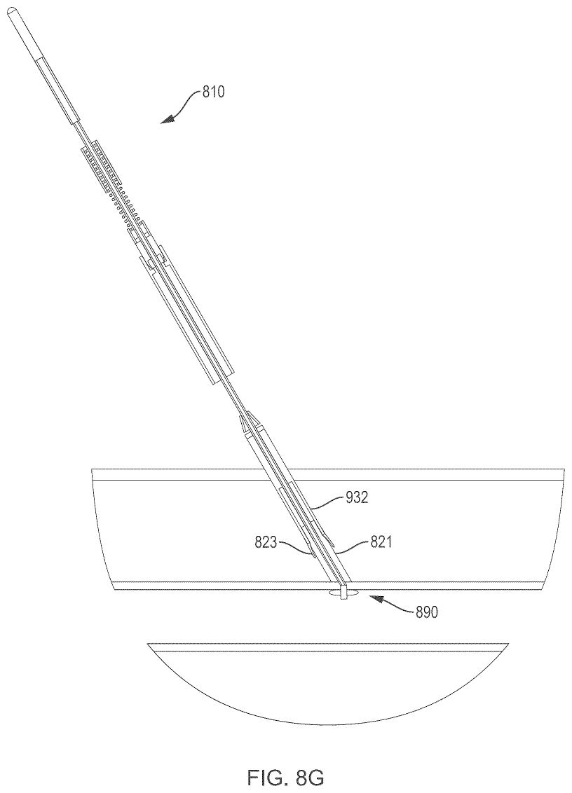

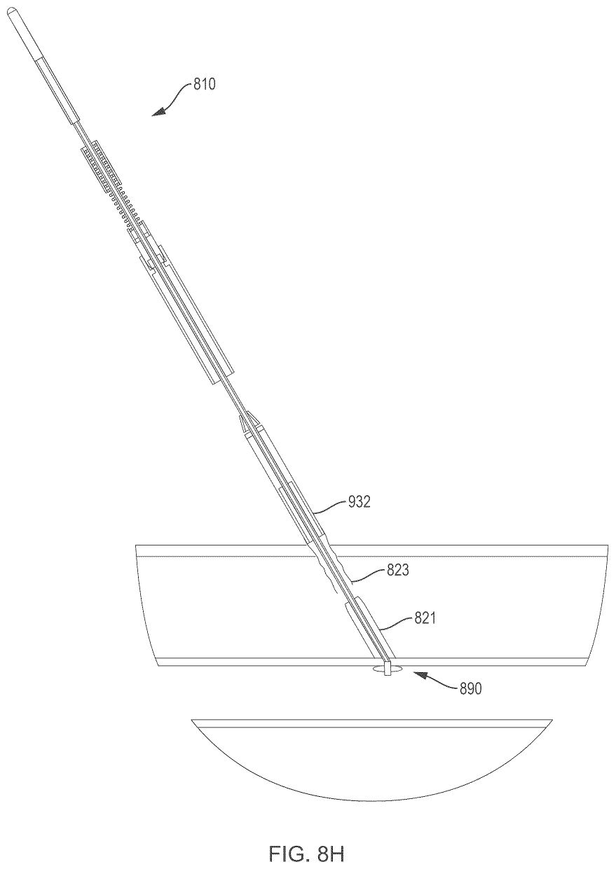

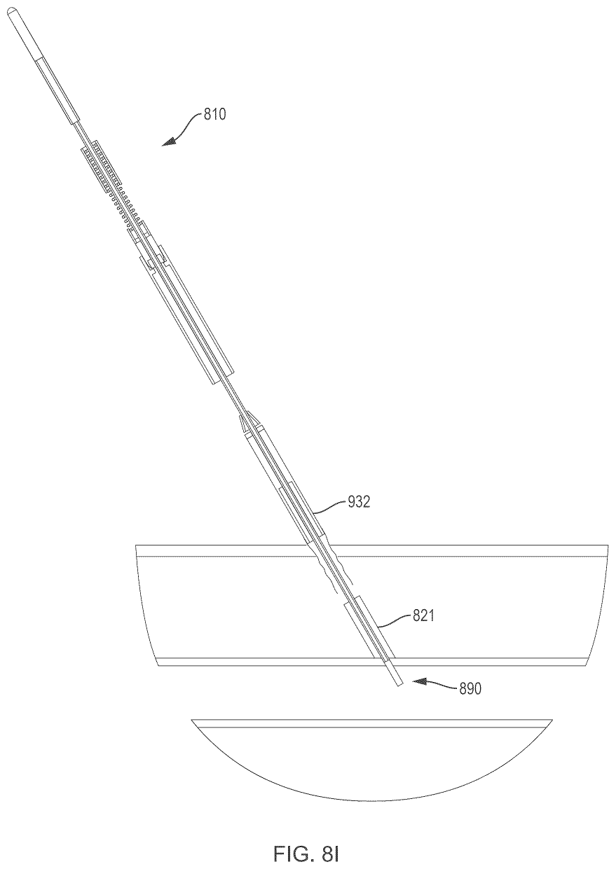

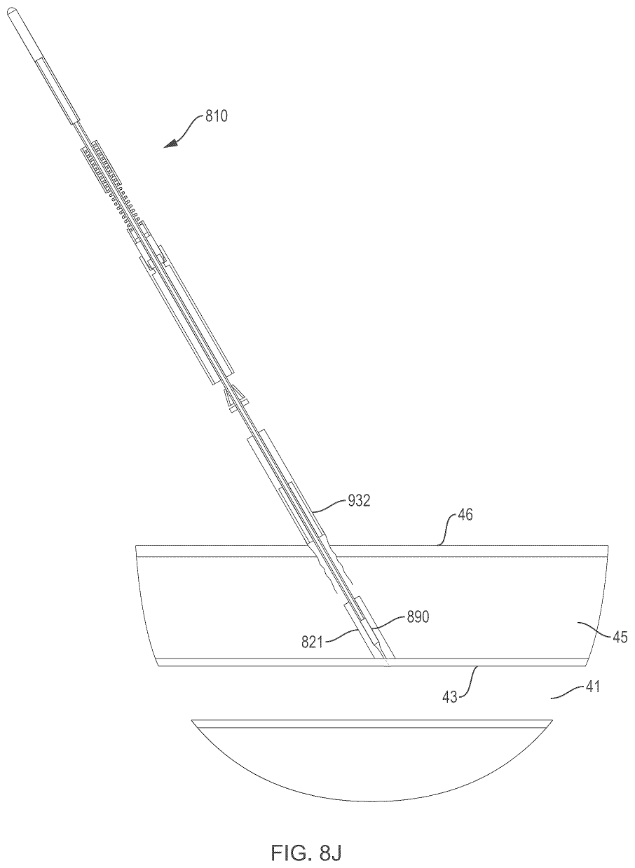

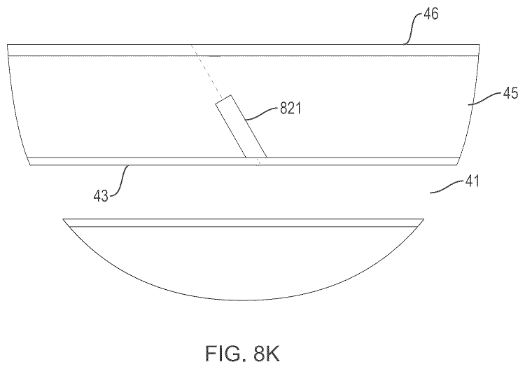

FIGS. 8A-8K are sectional views showing the use of a sealing apparatus 810 with a vascular introducer sheath 840, according to one embodiment of the present invention. In FIGS. 8A-8D, the introducer sheath 840 is inserted into a blood vessel lumen 41 and may then be used in the placement and deployment of a hemostatic implant.

The introducer sheath 840 is placed so as to project into a blood vessel lumen 41, so that the introducer sheath passes from the skin surface 46 through tissue 45 in a tissue tract. A vascular wall penetration 42 will thus be present in the vascular wall 43, as shown in FIG. 8A. The sealing apparatus 810 is then introduced through the introducer sheath 840 so that the expansible occlusion element 890 passes out through the distal end of the sheath, as shown in FIG. 8B. The handle assembly 878 of the sealing apparatus 810 will remain outside of the sheath and accessible to the user so that the slide assembly 801 may be pulled relative to the cylindrical body 803 to radially expand the occlusion element 890, as shown in FIG. 8C, so that the occlusion element 890 is in its expanded state within the blood vessel. The vascular access sheath 840 may then be withdrawn over the exterior of the sealing apparatus 810 while the sealing apparatus is simultaneously pulled proximally to seat the expanded occlusion element 890 against the vascular penetration 42, as shown in FIG. 8D. Alternatively, the sealing apparatus 810 is pulled proximally, causing the expanded occlusion element 890 to be seated against the vascular penetration 42, and then the vascular access sheath 840 is withdrawn over the exterior of the sealing apparatus 810 to arrive at configuration shown in FIG. 8D.

At that point, the retractable member 932 and key 826 are available for manipulation by the user. The key 826 may then be distally advanced over the outer tube 871 so that the key engages and depresses the latch 820 as illustrated in FIG. 8E. The key 826 and retractable member may then be manually pulled in a proximal direction over the outer tube 871 to release the hemostatic implant 821, as shown in FIGS. 8F-8H.

A thin, flexible, peelable sleeve 823 surrounds the hemostatic implant 821 throughout the steps shown in FIGS. 8B-8E. The distal end of this sleeve 823 is connected to the distal end of the retractable member 932. As the retractable member 932 is pulled in a proximal direction, the distal end of the sleeve 823 is pulled away from the hemostatic implant 821 and in a proximal direction, as shown in FIGS. 8F and 8G. As the sleeve is peeled away from the hemostatic implant 821, more and more of the implant is exposed to the surrounding tissue. The hemostatic implant 821 absorbs water from the surrounding tissue and begins expanding. This process is performed quickly so as to avoid the hemostatic implant 821 expanding so early that it might become stuck within the retractable member 932 before the retractable member is fully retracted away from the hemostatic implant.

At the stage shown in FIG. 8H, the peelable sleeve 823 is fully pulled off of the hemostatic implant 821, which is now fully exposed to the surrounding tissue and continues to expand. In FIG. 8I the occlusion element 890 is contracted in preparation of being withdrawn through the hemostatic implant 821 and the tissue tract.

In FIG. 8J the contracted occlusion element 890 passes through the hemostatic implant 821, as the sealing apparatus 810 is being removed proximally through the tissue tract. In FIG. 8K the sealing apparatus, except for the hemostatic implant 821, has been fully removed from the tissue tract. As a result, the hemostatic implant 821 has been placed against the blood vessel wall 43 in a position to inhibit blood from escaping the blood vessel into the tissue tract, thereby reducing or eliminating the need to apply pressure on the tissue tract.

The hemostatic implant 821, which may optionally carry anti-proliferative, coagulation-promoting, and/or radiopaque substances, will remain in place inhibiting bleeding through the upper portions of the tissue tract and allowing the vascular wall penetration to heal. Over time, the hemostatic implant 821 will preferably biodegrade, leaving a healed tissue tract and vascular wall penetration which are usually suitable for re-entry at a subsequent time.

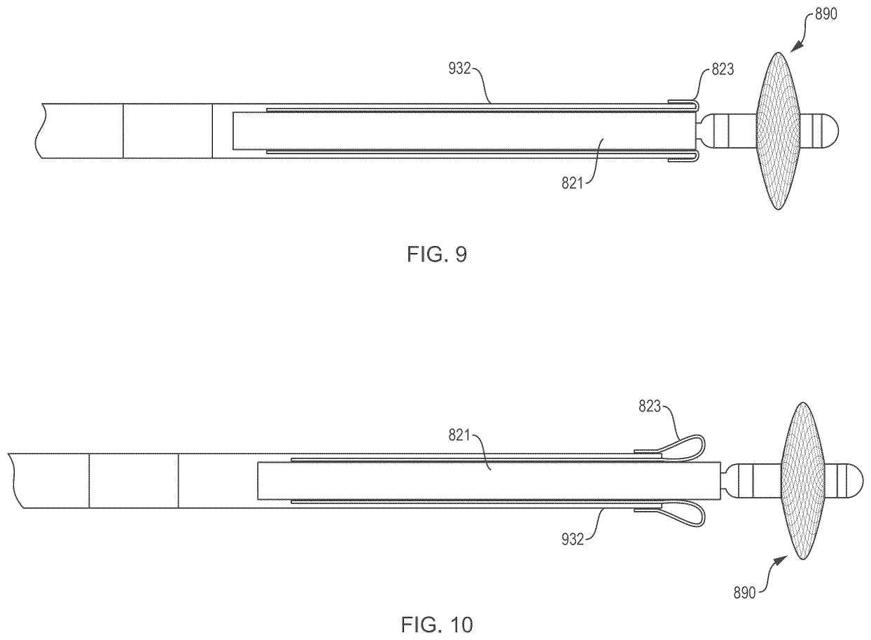

FIGS. 9 and 10 show a peelable sleeve 823, the distal end of which is being pulled by a retractable member 932. FIG. 9 shows the arrangement of the peelable sleeve 823, the retractable member 932, and the hemostatic implant 821 at the very beginning of the peeling process. The retractable member 932 shown in FIG. 9 covers the outer perimeter of the hemostatic implant 821. The peelable sleeve 823 is connected to distal end of the retractable member 932 and is located between retractable member 932 and the hemostatic implant 821. As shown in FIGS. 9 and 10, the occlusion element 890 remains in a radially expanded configuration during the peeling process. During the peeling process, the radially expanded occlusion element 890 remains seated against the inner surface of the blood vessel, so as to inhibit flow into the tissue tract.

FIG. 10 shows, a short moment later, the hemostatic implant 821 being exposed at its distal end to the surrounding tissue, as the sleeve 823 is being peeled in a proximal direction and away from the outer surface of the hemostatic implant 821. In the prior system, the slidable protective sleeve is pulled on its proximal end, and the inner surface of the prior, slidable protective sleeve rubs against the outer surface of the hemostatic implant. This rubbing motion in the prior system applies a frictional force in an axial proximal direction against the hemostatic implant.

The peelable sleeve 823 in FIGS. 9 and 10 is pulled from its distal end in such a way that the peelable sleeve is retracted in a peeling manner. This peeling motion does not cause rubbing against outer surface of the hemostatic implant. The peeling causes a force vector in an outwardly traverse direction away from the hemostatic implant.

By peeling the sleeve away from the hydratable hemostatic implant, the amount of friction-especially friction causing a force vector in an axial, proximal direction is reduced, thereby reducing the proximal pull on the hemostatic implant. As a result, the implant remains even closer to vascular wall as the sealing apparatus is proximally withdrawn from the tissue tract.

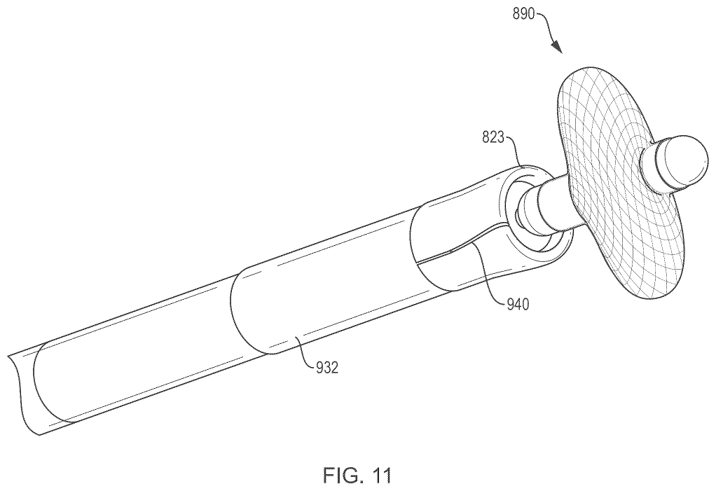

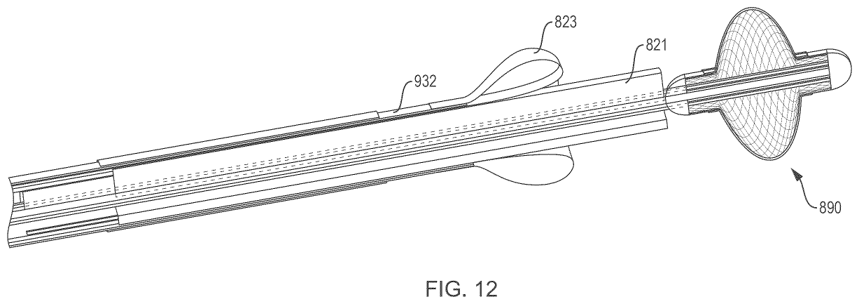

FIG. 11 is a perspective detail view of an apparatus corresponding to the arrangement shown in FIG. 9. FIG. 12 is a cut-away perspective view of an apparatus corresponding to the arrangement shown in FIG. 10. To improve the sleeve's peeling characteristics, the sleeve includes a longitudinal slit, or two slits, or more. Alternatively, longitudinally arranged perforations may be used in lieu of slits. FIG. 11 shows one longitudinal slit 940 in the sleeve 823. Preferably, the sleeve 823 includes a second slit on the side facing away from the viewer of FIG. 11.

As in the prior system described hereinabove in connection with FIGS. 1-7, the sealing apparatuses of FIGS. 8A-8K and FIGS. 9-12 may further include a biodegradable plug that, prior to retraction of the sleeve, protects the hydratable hemostatic implant from exposure to blood or other body fluids when present in the tissue tract. The plug may be composed of any of the materials discussed previously. The plug works to block or inhibit passage of significant amounts of fluids to the hydratable hemostatic implant. Once the sleeve begins being peeled away, the plug will quickly dissolve in the body fluids, becoming resorbed by the tissue over a relatively short time frame. In contrast, the swollen hemostatic implant, which is preferably made of collagen, will not dissolve and will be resorbed only slowly over time in order to provide the desired hemostatic effect.

While the above is a complete description of the preferred embodiments of the invention, various alternatives modifications, and equivalents may be used. Therefore, the above description should not be taken as limiting the scope of the invention which is defined by the appended claims.

The embodiments of the invention described above are intended to be merely exemplary; numerous variations and modifications will be apparent to those skilled in the art. Such variations and modifications are intended to be within the scope of the present invention as defined by any of the appended claims.

What is claimed is:

1. An apparatus for sealing a blood vessel wall penetration disposed at an end of a tissue tract, said apparatus comprising:
a shaft having a proximal end and a distal end;
a hemostatic implant disposed over or adjacent to an exterior surface of the shaft proximal to the distal end, said hemostatic implant being hydratable to expand to occlude the tissue tract when exposed to body fluids within the tissue tract; and
a sleeve peelably disposed over an outer surface of the hemostatic implant and proximal to the distal end.

2. The apparatus of claim 1, further including a retractable member, wherein the retractable member is attached to a distal end of the sleeve, such that when the retractable member is retracted in a proximal direction, the sleeve is peeled away from the outer surface of the hemostatic implant.

3. The apparatus of claim 2, wherein the retractable member is cylindrical and surrounds the hemostatic implant and the sleeve, such that as the retractable member is retracted in a proximal direction the hemostatic implant and the sleeve are exposed.

4. An apparatus for sealing a blood vessel wall penetration disposed at an end of a tissue tract, said apparatus comprising:
a shaft having a proximal end and a distal end;
an occlusion element near the distal end of the shaft;
a hemostatic implant disposed over or adjacent to an exterior surface of the shaft proximal to the occlusion element, said hemostatic implant being hydratable to expand to occlude the tissue tract when exposed to body fluids within the tissue tract; and
a sleeve peelably disposed over an outer surface of the hemostatic implant and proximal to the occlusion element;
wherein peeling of the sleeve exposes the hemostatic implant such that the hemostatic implant expands and occludes the tissue tract.

5. The apparatus of claim 4, further including a retractable member, wherein the retractable member is attached to a distal end of the sleeve, such that when the retractable member is retracted in a proximal direction, the sleeve is peeled away from the outer surface of the hemostatic implant.

6. The apparatus of claim 5, wherein the retractable member includes at a ring at a distal end of the retractable member.

7. The apparatus of claim 5, wherein the retractable member is cylindrical and surrounds the hemostatic implant and the sleeve, such that as the retractable member is retracted in a proximal direction the hemostatic implant and the sleeve are exposed.

8. The apparatus of claim 5, wherein the sleeve includes a longitudinal slit.

9. The apparatus of claim 5, wherein the sleeve includes a plurality of longitudinal slits.

10. The apparatus as in claim 4, wherein the occlusion element is shiftable between a radially contracted configuration for passage through the tissue tract and a radially expanded configuration for deployment within the blood vessel to occlude the penetration.

11. The apparatus as in claim 10, further comprising a back stop on the shaft, wherein the back stop engages the hemostatic implant to immobilize the implant while the sleeve is being peeled from the implant and the contracted occlusion element is being withdrawn past the implant.

12. The apparatus as in claim 10, wherein the shaft comprises an outer tube and an inner rod, and wherein the occlusion element has a distal end connected to a distal end of the rod and a proximal end connected to a distal end of the tube so that proximal retraction of the rod relative to the tube effects radial expansion of the occlusion element and distal advancement of the rod relative to the tube effects radial contraction of the occlusion element, wherein the occlusion element comprises a braided mesh covered by an elastic membrane.

13. The apparatus as in claim 4, wherein the hemostatic implant comprises a body which circumscribes the shaft.

14. The apparatus as in claim 4 wherein the hemostatic implant comprises a body which is configured to open and expand away laterally from the shaft.

15. The apparatus as in claim 4, wherein the hydratable hemostatic implant comprises a biodegradable polymer selected from the group consisting of polyethylene glycols, collagens, and gelatins.

16. The apparatus as in claim 4, wherein the hemostatic implant comprises an active agent which comprises an anti-proliferative agent selected from the group consisting of sirolimus and paclitaxel, or a procoagulant selected from the group consisting of thrombin and tissue factor.

17. A method for sealing a blood vessel penetration disposed at the end of a tissue tract, the method comprising:
providing an apparatus including a shaft, a hemostatic implant disposed on an exterior surface of the shaft, and a sleeve covering an outer surface of the hemostatic implant;
introducing the shaft through the tissue tract to position the hemostatic implant within the tissue tract, wherein the hemostatic implant is covered by the sleeve while the shaft is being introduced;
peeling the sleeve to expose the hemostatic implant to the tissue tract, wherein the hemostatic implant expands upon direct contact with the tissue tract; and
withdrawing the shaft past the expanded hemostatic implant which remains in the tissue tract.

18. The method as in claim 17, wherein the apparatus further includes an occlusion element and the method further comprises deploying the occlusion element to inhibit blood flow from the blood vessel into the tissue tract.

19. The method as in claim 18, wherein deploying the occlusion element comprises shifting the occlusion element between a radially contracted configuration for passage through the tissue tract and a radially expanded configuration for deployment within the blood vessel to occlude the penetration.

20. The method as in claim 17, wherein the hemostatic implant is prevented from being displaced proximally by a back stop on the shaft while the sleeve is peeled and while the shaft is withdrawn.

21. The method as in claim 17, wherein the apparatus further includes a retractable member, wherein the retractable member is attached to a distal end of the sleeve, such that when the retractable member is retracted in a proximal direction, the sleeve is peeled away from the outer surface of the hemostatic implant.

22. The apparatus of claim 21, wherein the retractable member includes at a ring at a distal end of the retractable member.

23. The apparatus of claim 21, wherein the retractable member is cylindrical and surrounds the hemostatic implant and the sleeve, such that as the retractable member is retracted in a proximal direction the hemostatic implant and the sleeve are exposed.

24. A method for sealing a blood vessel penetration disposed at the end of a tissue tract, the method comprising:
providing an access sheath passing through the tissue tract from a skin surface to a blood vessel;
providing a sealing apparatus including a shaft, a hemostatic implant disposed on an exterior surface of the shaft, a sleeve covering an outer surface of the hemostatic implant, and an expandable occlusion element;
placing the sealing apparatus within the access sheath and positioning the sealing apparatus within the tissue tract so that the sealing apparatus protrudes into the blood vessel, wherein the hemostatic implant is covered by the sleeve while the sealing apparatus is within the access sheath and being introduced into the tissue tract;
after introducing the sealing apparatus through the tissue tract, expanding the occlusion element within the blood vessel, seating the expanded occlusion element against a wall of the blood vessel at the end of the tissue tract to inhibit blood flow from the blood vessel into the tissue tract, and removing the access sheath;
after removing the access sheath, peeling the sleeve away from the hemostatic implant to expose the hemostatic implant to the tissue tract, wherein the hemostatic implant expands upon direct contact with the tissue tract;
after exposing the hemostatic implant to the tissue tract, contracting the occlusion element; and
after contracting the occlusion element, withdrawing the shaft and the contracted occlusion element past the expanded hemostatic implant, which remains in the tissue tract.

25. The method as in claim 24, wherein the sealing apparatus further includes a retractable member, wherein the retractable member is attached to a distal end of the sleeve, such that when the retractable member is retracted in a proximal direction, the sleeve is peeled away from the outer surface of the hemostatic implant.

26. The apparatus of claim 25, wherein the retractable member is cylindrical and surrounds the hemostatic implant and the sleeve, such that as the retractable member is retracted in a proximal direction the hemostatic implant and the sleeve are exposed.
